(19) United States
(12) Patent Application Publication
(10) Pub. No.: US 2021/0139942 A1
(43) Pub. Date: May 13, 2021

(54) DROPLET-BASED MONITORING OF BIOLOGICAL SAMPLES

(71) Applicant: Purdue Research Foundation, West Lafayette, IN (US)

(72) Inventors: SeyedehAida Ebrahimi, West Lafayette, IN (US); Muhammad Ashraful Alam, West Lafayette, IN (US)

(73) Assignee: Purdue Research Foundation, West Lafayette, IN (US)

(21) Appl. No.: 16/864,048

(22) Filed: Apr. 30, 2020

Related U.S. Application Data
(63) Continuation of application No. 15/269,972, filed on Sep. 19, 2016, now abandoned.
(60) Provisional application No. 62/220,656, filed on Sep. 18, 2015.

(51) Int. Cl. C12Q 1/04; G01N 27/12; G01N 15/06

(52) U.S. Cl. CPC C12Q 1/04; G01N 15/0656; G01N 27/12

(57) ABSTRACT

A method and apparatus for electrically monitoring a time-varying liquid droplet whose conductivity is continuously modulated by osmoregulation response of cells. According to the method, the droplet impedance or conductance is monitored over time as the droplet shrinks due to evaporation. The monitoring data is then compared to calibration data which is obtained by monitoring a reference droplet. The result of the comparison is then used to determine the concentration of viable (live) biological material contained in the droplet.

### CROSS-REFERENCE TO RELATED APPLICATIONS

The present patent application is a continuation of U.S. patent application Ser. No. 15/269,972, filed Sep. 19, 2016, which is related to and claims the priority benefit of U.S. Provisional Patent Application Ser. No. 62/220,656, filed Sep. 18, 2015, the contents of which is hereby incorporated by reference in its entirety into the present disclosure.

### TECHNICAL FIELD

The present disclosure relates to biological testing, and more specifically, electrical sensing of biological material.

### BACKGROUND

Rapid analysis of viability of a few bacterial cells in food, water, and/or clinical samples is critically important in a variety of fields, such as bioscience research, medical diagnosis, and hazard analysis in food industry. Under a microscope, bacteria cells are amazingly alive and perform a whole host of physiological functions, namely, multiplication through cell division, searching for resources by chemotaxis, controlling their water pressure by exchange of ions (through osmoregulatory system), etc. And yet, since the introduction of the plate counting method almost 130 years ago, the viability assays (e.g. impedance microbiology and fluorescence staining) continue to rely only on cell multiplication as the exclusive physiological process to differentiate between dead and live cells. Unfortunately, cell-division time can vary from hours to weeks depending on the bacteria type (10-20 min for Escherichia coli vs. 15-16 hours for Mycobacterium Tuberculosis). Such type-dependence prevents the possibility of real-time detection of cell concentration by means of growth-based techniques, especially at low concentration. Therefore, improvements are needed in the field.

### SUMMARY

The present disclosure provides a method and apparatus for electrically monitoring a time-varying liquid droplet whose conductivity is continuously modulated by osmoregulation response of cells. According to the method, the droplet impedance or conductance is monitored over time as the droplet shrinks due to evaporation. The monitoring data is then compared to calibration data which is obtained by monitoring a reference droplet. The result of the comparison is then used to determine the concentration of viable (live) biological material contained in the droplet.

According to one aspect, a method of determining a concentration and viability of a biological material in a liquid sample is disclosed. The method involves electrical monitoring of a liquid droplet whose conductivity is modulated over time by osmoregulation response of cells contained within the droplet. Concentration and viability of the cells are then determined by monitoring the time-dependent data, and comparing the monitored data to calibration data in order to determine the concentration of the cells.

According to another aspect, a device for determining a concentration of a biological material in a liquid sample is disclosed, comprising a first electrode and a second electrode. The first and second electrode are configured to pin a liquid droplet in a first contact area such that as the droplet evaporates, the contact area remains substantially constant. A monitoring unit is operatively connected to the first and second electrodes. The monitoring unit is configured to electrically monitor the droplet to determine monitoring data as conductivity of the droplet is modulated over time by osmoregulation response of cells contained within the droplet.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1(a) shows a schematic diagram of a biological sample with cells in a growth medium.

FIG. 1(b) shows the cells from the sample resuspended in deionized (DI) water.

FIG. 1(c) shows droplet-based conductance measurements as a function of frequency on droplets containing 1-5×10⁸ cells/ml of live and dead cells.

FIG. 1(d) shows time-averaged relative conductance wrt the analyte-free (reference) solution.

FIG. 2(a) shows time-variation of the turgor pressure (ΔΠ) as the cells go through the conditions shown in FIG. 1(a).

FIG. 2(b) shows the per-cell conductivity (σ*) as cells go through the steps shown in FIG. 1.

FIG. 3(a) shows measured conductance, G(t*), as a function of the normalized evaporation time for live samples.

FIG. 3(b) shows measured conductance, G(t*), as a function of the normalized evaporation time for heat-treated samples.

FIG. 3(c) shows extracted per-cell conductivities (σ*). The symbols show σ*_{l,i/f} and σ*_{d,i/f} values obtained from FIGS. 3(a) and 3(b), respectively. These curves act as calibration curves for determination of ρₜₒₜ and a of an unknown sample. The lines depict σ*_{l,i/f} and σ*_{d,i/f} fitted with σ*≈aρₜₒₜ^{b}, with a and b as the fitting parameters.

FIG. 4(a) shows the linear relation between the estimated cell concentration, ρ_{estim.}, obtained using the technique of the present disclosure, vs. the actual value, ρₜₒₜ, for seven different samples.

FIG. 4(b) shows the linear relation between estimated cell ratio, α_{estim.} Obtained using the technique of the present disclosure, vs. the actual value, α, for seven different samples.

FIG. 5(a) shows the differential conductance G(t*) with respect to reference solutions with different ionic concentration and therefore different Πₒᵤₜ (TM and TM×10) as a function of time for cell concentration of 10⁷ cells/ml and α=1.

FIG. 5(b) shows the corresponding extracted σ values at t*ᵢ and t*_{f}, with σ*_{l,i/f}^{TM×10}<σ*_{l,i/f}^{TM}, which is due to the initially larger Πₒᵤₜ in solution with TM×10 than TM (arrow 501). σ*ᵢ>σ*_{f} as described herein, and shown by the (ii) process in FIG. 3c (arrow 502).

FIG. 6a shows time-varying conductance of various types of cells according to one embodiment.

FIG. 6b shows the ratio of the final to the initial per-cell conductivities for the cell types shown in FIG. 6a.

FIG. 7 shows the detection time vs. bacterial cell concentration reported by prior art viability assays, including label-free methods such as, DEP-based, on-chip IM with and without DEP-based cell captured, macroscale IM, wireless biosensor based on shift in the resonance frequency (Δfᵣₑₛ), and molecular-based methods, such as LAPS for detection of mRNA, as compared to the presently disclosed method and apparatus.

### DETAILED DESCRIPTION

When exposed to osmotic shocks, bacteria survive by regulating the osmotic pressure difference across their cell envelope. The pressure difference (also known as turgor pressure) is defined by ΔΠ [symbol] Π_{cyt}−Πₒᵤₜ, where Π_{cyt} and Πₒᵤₜ are the cytoplasm and external osmotic pressures, respectively. The turgor pressure is regulated through activation of specific ‘emergency valves’, which rapidly modulate the concentration of the solutes (including ionic species) in both the external and cytoplasmic solutions.

When the turgor pressure ΔΠ increases above the natural turgor pressure (under osmotic downshift), mechanosensitive (MS) channels in the bacteria open to release intracellular osmolytes to the surrounding medium within fractions of a second. These proteins, which in the case of Escherichia coli are majorly MscL and MscS channels, pump out different osmolytes (including ions, ATP, lactose, etc.) into the surrounding medium, without any damage to the cell envelope and/or lysis. In contrast, upon osmotic upshift, another group of osmoregulatory transporters are activated by the bacteria to restore the natural turgor pressure (e.g. by uptaking solutes from the surrounding medium).

The present disclosure utilizes osmoregulation, which is equally universal as cell multiplication but much faster, as an effective, real-time monitor of bacteria cells. In one embodiment, bacteria cells are confined in a liquid droplet placed on an impedance sensing unit. The droplet forms a tunable, precisely controlled microenvironment for bacteria cells. As the droplet evaporates, the analytes are forced toward the sensor surface, instead of freely diffusing. Evaporation-induced beating of the diffusion limit, results in much higher sensitivity and shorter response time of the presently disclosed droplet-based sensor compared to the classical impedance sensors. Upon evaporation, concentration of the droplet's solutes increases, and correspondingly, so does its osmotic pressure. This dynamic environment stimulates the osmoregulatory system of live cells, resulting in uptake (‘stealing’) of osmolytes from the external environment and ‘hiding’ them inside the cells. Therefore, while the droplet conductance increases with evaporation (due to the increased ion concentration), the presence of bacteria suppresses the net increase by shielding a fraction of ions away from the electric field. An elementary theoretical model, to be discussed below, explains the results consistently. In addition, as a reference, another group of cells that lost their osmoregulation ability due to heat-treatment, and therefore are ‘osmoregulatory-dead’ (or simply defined as ‘dead’) were analyzed. Use of osmoregulation in conjunction with the droplet-based impedance sensor, provides selective differentiation of live and dead cells down to ˜10⁴ cells/ml and is achievable within 20 minutes. Further, the osmoregulatory response of most bacteria types has similar time-scale, therefore, the detection time of the present assay is anticipated to be less dependent on the bacteria type. In contrast, as mentioned above, growth-based techniques require hours to weeks to numerate cells (depending on the bacteria type). The presently disclosed method can be used in tandem with existing growth-based protocols to further improve the sensitivity and selectivity, with the corresponding trade-off in detection time. Moreover, the presently disclosed method may also be used as a non-destructive (as opposed to patch-clamp methods), indirect characterization tool to fingerprint cells' osmoregulatory response to their environment.

FIGS. 1a-1d illustrates a process and apparatus for determining the amount of viable bacteria according to one embodiment. First, to prepare live samples with fully-functional osmoregulatory system, cells 102 are incubated in a growth medium 104 under appropriate growth conditions and resuspended in deionized (DI) water. This step is performed to eliminate the parasitic effects of the growth medium on the conductance signal. As shown in FIG. 1a, at time t*₀, which is when the cells 102 are initially resuspended in DI water 105, the MS channels 106 open to efflux the intracellular content (shown by arrows 107), and eventually restore the initial turgor pressure ΔΠ₀ (time t*_{MS}). This process (opening and closing within t*_{MS}) is very fast and happens within a fraction of a second. As a reference, osmoregulatory-dead (‘dead’) cells are then prepared by heating an aliquot of live samples at 80° C. for 20 min in one example. In this case, the cells are intentionally not lysed, but instead their protein channels just damaged by heat to impair cells' ability to osmoregulate. Live and dead samples at various cell concentrations are then prepared by serial dilution before impedance measurements. As shown in FIG. 1b, an impedance sensor 110 is provided which comprises at least two electrode sets 112 and 114, which, in one embodiment, each electrode set comprises an array of Ni micro-fins with superhydrophobic properties. The design of such multifunctional hydrophobic electrodes may be configured for creating droplets with highly reproducible geometric shape, which provides high precision measurement of dynamic impedance of evaporating droplets. The sensor 110 may further comprise an electronic monitoring unit having a processor and a memory, and configured to monitor the impedance of a droplet 116 through a connection to electrode sets 112 and 114. In certain embodiments, wherein at least three electrodes are provided, the system may be configured to electrically monitor the droplet using various selected pairs of the electrodes to map the physical distribution of said cells within the droplet. For example, if there are N electrodes, and therefore N(N−1)/2 pair of electrodes to obtain signal from, each pair monitors the droplet from different physical vantage points. The received signals may then be used electrically map the physical distribution of the biomolecules.

In one embodiment, droplets 116 (which in one example have a 3 μl volume, although smaller or larger volumes may be used, for example in the range of 1 μl-10 μl) are deposited on the sensor surface 115 as shown in FIG. 1b and their conductance is monitored as they evaporate from time t*ᵢ to t*_{f}. As time passes, the droplet size reduces which causes the ionic concentration ρₒᵤₜ to increase. This evaporation-induced increase causes the turgor pressure ΔΠ to decrease. Decrease of ΔΠ activates the osmoregulatory transporters of live cells 109 to uptake ions from the droplet solution (indicated by single-sided arrows 108). In case of dead cells 111 with disintegrated proteins, the ions can diffuse freely in both directions (indicated by double-sided arrows 113). FIG. 1c shows droplet-based conductance measurements G(t*), as a function of frequency on droplets containing 1-5×10⁸ cells/ml of live and dead cells according to one example. Time-averaged conductance signals for live and dead samples (ΔYₗ and ΔY_{d}) with cell concentration ranging from 10⁴ to 10⁷ cells/ml are plotted in FIG. 1d. As can be seen (i) samples with dead cells generate larger electrical conductance and (ii) conductance increases with cell concentration, ρₜₒₜ. ΔY is defined as the time-averaged relative conductance with respect to the analyte-free (reference) solution at various total cell concentrations. ΔYₗ and ΔY_{d} are obtained for live and dead samples, respectively. The error bars are the SDs from sample-average with k=9. Averaging is over nine data points obtained during evaporation. Because of the time-multiplexing capability of the approach, the error bars are comparable to the symbol size. Inset of FIG. 1d shows the two measurements of a given sample: conductance measurement on the as-prepared sample gives Gₘ(t*) and consequently, ΔYₘ*. Additionally, a postheating step is carried out to deactivate all cells and pin α to zero. The postheating step results in the upshift of the signal (ΔYₘ* to ΔYₕ*). We estimate ρₜₒₜ by intersecting ΔYₕ* and the ΔY_{d} curve as schematically shown. The estimated value is denoted by ρₜₒₜ*.

As schematically illustrated in FIG. 2a, when cells 102 (either live cells 109 or dead cells 111) are initially resuspended in DI water at time t*, Πₒᵤₜ=Π_{DI}˜0. As a result, the cells 102 experience a significant turgor increase beyond the natural turgor value, ΔΠ₀. To restore the natural turgor, the MS channels in the live cells 109 open up and pump out the cytoplasmic osmolytes, including ionic entities. The gating process (opening and closing of the MS channels) and restoring of ΔΠ₀ is completed in less than a milli-second (by t*_{MS}). Release of ions from cytoplasm to the external solution results in a conductance increase as compared to an analyte-free reference.

FIG. 2a shows the time-variation of the turgor pressure (ΔΠ) as the cells 102 go through the conditions shown in FIG. 1. The insets of FIG. 2a show a droplet 202 as it evaporates from time t*ᵢ to t*_{f}. When exposed to an increase in the osmotic pressure of their external environment (Πₒᵤₜ), e.g. due to evaporation from t*ᵢ to t*_{f} in FIG. 2a, bacteria usually respond by uptake of osmolytes, either from the environment or by synthesis. The most rapid response of cells to the decrease of ΔΠ (below ΔΠ) is by uptake of K+ ions from the environment via turgor-responsive transport systems, such as TrK transporters in E. coli and Salmonella. The uptake of ions from the droplet 202 is reflected in an effective decrease in ionic contribution of each cell (defined as per-cell conductivity σ*) with time, as schematically shown by the solid curve 210 in FIG. 2b. Such ‘stealing’ of ions from the droplet by the viable or live cells results in a suppressed conductance modulation over time as compared to the scenario where cells are irresponsive to the modulation of osmotic pressure (represented by the almost constant dotted line 212 in FIG. 2b). In that case (dead cells), evaporation would have been the only decisive factor in overall increase of conductance (G) over time due to continuous amplification of the droplet's ionic concentration (ρₒᵤₜ).

It is known that conductance generally increases with cell concentration. With respect to live samples, ion- (more precisely osmolyte-) release from bacterial cells in a hypotonic solution (when ΔΠ>ΔΠ₀) is the main reason for change of the solution impedance with cell concentration. For example, suspensions of Salmonella in DI water with different concentrations result in different impedance responses. Impedance of the cell suspensions decreases with increase of cell concentration (consistent with FIG. 1d). Although it has been suggested in the prior art that the change of impedance is due to the charges associated with cell wall and release of ions from cells, it has not been quantitatively confirmed. In this regard, the effective density of the species released to the solution can be calculated through the correlation between osmotic pressure and concentration of the osmolytes. It is estimated that after sample preparation and reaching the equilibrium (from t*_{MS} to t*ᵢ), a sample with ˜3×10⁸ cells/ml causes a conductance increase of Gₗ^{calc}˜3.4-5.2 μS. Remarkably, this simple estimate is in excellent agreement with the measured value Gₗ^{exp}=2.56 μS.

With respect to dead or heat-treated samples, cell envelope becomes permeable, and there will be no barrier for the solutes to diffuse across. Therefore, the intracellular content of cells, including ions (K⁺, Na⁺, Mg²⁺), DNA, RNA, amino acids, and enzymes, leak to the surrounding environment. As a result, the solution conductance increases significantly, more so than the live samples. This increase is proportional to the number of cells in a given volume. By assuming that nearly all the cytoplasmic content is released to the surrounding solution upon heat treatment, a sample with ˜3×10⁸ cells/ml results in a conductance increase of around G_{d}^{calc}˜6.6-9 μS, which is consistent with the measured value G_{d}^{exp}=6.34 μS.

In reality, the samples under study may contain a mixture of live and dead cells. Therefore, the ability to distinguish between them is of critical importance for practical applications. Below, a simple, yet comprehensive, conductance model of a droplet containing a mixture of cells is provided. Then, the model is validated by the experimental data and it is demonstrated that the approach can determine, with a high precision, fraction of live cells in a mixture of dead and live ones.

Droplet modeling reveals that cells ‘steal’ ions over time. In a given sample, the number of live and dead cells are nₗ (ρₗ≡nₗ/V₀) and n_{d} (ρ_{d}≡n_{d}/V₀), respectively, with V₀ being the initial droplet volume. The ratio of live cells to the total number of cells is hence

$$α ≡ \frac{{ρ}_{l}}{{ρ}_{t o t} ( = {ρ}_{l} + {ρ}_{d )}} = \frac{{n}_{l}}{{n}_{tot} ( = {n}_{l} + {n}_{d )}} ,$$

where ρₜₒₜ≡nₜₒₜ/V₀. Then, Eq. 1 is derived by using the conductance formulation for an evaporating droplet (Eq. S3 in SI) and defining the per-cell conductivities as σ*_{l/d}(t*) [symbol] [symbol] μ [symbol] R_{l/d}(t*). [symbol] μ [symbol] and R_{l/d}(t*) are the effective mobility and the number of released ions from individual live/dead cells, respectively.

$$G ( {t}^{*} ) = ( {σ}_{l} ( {t}^{*} ) + {σ}_{d} ( {t}^{*} ) ) \frac{{H}_{Z}}{g ( {t}^{*} )} = \frac{{V}_{0} {H}_{Z} {ρ}_{t o t}}{V ( {t}^{*} ) g ( {t}^{*} )} [ α {σ}_{l}^{*} ( {ρ}_{t o t} , {t}^{*} ) + ( 1 - α ) {σ}_{d}^{*} ( {ρ}_{t o t} , {t}^{*} ) ] , \qquad [ 1 ]$$

Here, H_{z} represents the time-invariant length of the deposited droplet.

Based on Eq. 1, the initial conductance (G_{i,α} at t*ᵢ) and final conductance (G_{f,α} at t*_{f}) would be

$$\begin{matrix} {G}_{i , α} = {ρ}_{tot} \frac{1}{C 1} [ {ασ}_{l , i}^{*} + ( 1 - α ) {σ}_{d , i}^{*} ] & [ 2 ] \\ {G}_{f , α} = {ρ}_{tot} \frac{1}{C 2} [ {ασ}_{l , f}^{*} + ( 1 - α ) {σ}_{d , f}^{*} ] \\ {V}_{0} \hat{=} V ( {t}_{i} ) , {g}_{0} \hat{=} g ( {t}_{i}^{*} ) , {V}_{f} \hat{=} V ( {t}_{f}^{*} ) , {g}_{f} \hat{=} g ( {t}_{f}^{*} ) , C 1 \hat{=} \frac{{g}_{0}}{{H}_{z}} , C 2 \hat{=} \frac{{g}_{f}}{{H}_{z}} \frac{{V}_{f}}{{V}_{0}} , {σ}_{l , i}^{*} \hat{=} {σ}_{l}^{*} ( {ρ}_{tot} , {t}_{i}^{*} ) , {σ}_{l , f}^{*} \hat{=} {σ}_{l}^{*} ( {ρ}_{tot} , {t}_{f}^{*} ) , {σ}_{d , i}^{*} \hat{=} {σ}_{d}^{*} ( {ρ}_{tot} , {t}_{i}^{*} ) , {σ}_{d , f}^{*} \hat{=} {σ}_{d}^{*} ( {ρ}_{tot} , {t}_{f}^{*} ) & [ 3 ]\end{matrix}$$

Time-dependent conductance values for samples with all-alive (α=1) and all-dead (α=0) cells are plotted in FIGS. 3a and 3b. By inserting G_{i,1}, G_{f,1}, G_{i,0}, and G_{f,0} (indicated by arrows) into Eq. 2 and 3, we extracted σ*_{l,i}, σ*_{l,f}, σ*_{d,i}, and σ*_{d,f}, which are plotted in FIG. 3c. The lines in FIG. 3c represent the fitted curves with a power-law dependence of σ* on ρₜₒₜ (σ*≈a(a)ρₜₒₜ^{b(α)}).

The extracted values of σ* are used in estimation of ρₜₒₜ and α in seven different samples. For a given sample with measured initial and final conductance of G(α, ρₜₒₜ, t*ᵢ) and G(α, ρₜₒₜ, t*_{f}), the numerical solution of Eq. 2 and Eq. 3 results in ρ_{estim.} and αₑₛₜᵢₘ. (plotted in FIG. 4). Remarkably, the estimated values are in excellent agreement with the measurement results. These plots confirm that different responses of osmoregulatory-live and -dead cells to the dynamic microenvironment enable their identification with a high precision.

Further, it should be noted that the plots in FIG. 3c convey three important observations: (i) at all cell concentrations, less ions are released from live cells compared to dead ones (σ*_{l,i/f}<σ*_{d,i/f}), (ii) the number of ejected ions per cell, σ*_{l/d}(t*), decreases with time (σ*_{l/d,f}<σ*_{l/d,i}), and (iii) σ* decreases with ρₜₒₜ. These observations can be explained as follows:
(i) Since heat-treated cells have a permeable cell envelope, the number of ions released from individual heat-treated cells is higher than that of the live ones at all times, therefore σ*_{l,i/f}<σ*_{d,i/f}.
(ii) As the droplet evaporates, its ionic concentration (ρₒᵤₜ) increases. To explain the decrease of σ* over time in both live and dead samples, we discuss the two cases separately.
   Osmoregulatory-live cells: In this case, increase of ρₒᵤₜ causes the turgor pressure across the cell envelope (ΔΠ) to decrease below the natural pressure (ΔΠ₀). As a result, the solution becomes ‘hypertonic’ to cells, causing the osmoregulatory transporters to activate and uptake ions from the environment. This is effectively equivalent to cells decreasing their ion release to the surrounding, and therefore σ*_{l,i}<σ*_{l,f}.
   To confirm this important observation, we have performed an experiment with α=1 and ρₜₒₜ=10⁷ cell/ml resuspended in a different reference solution (TM×10), with 10-times higher ionic concentration than the one we used so far (TM). FIG. 5a plots the measured G(t*) values for a sample in TM×10 and another sample in TM, at otherwise identical conditions. From these results, we calculated σ*_{l,i} and σ*_{l,f} as previously explained, and plotted them in FIG. 5b. This plot shows that σ*_{l,i/f}^{TM×10}<σ*_{l,i/f}^{TM} suggesting ΔΠ_{TM×10}<ΔΠ_{TM}(<ΔΠ₀). This observation confirms our previous statement that when cells are suspended in a solution with higher ρₒᵤₜ (higher Πₒᵤₜ), they experience larger decrease of the turgor pressure, and need to steal more solutes from the environment to restore ΔΠ₀.
   Osmoregulatory-dead cells: In this case, the decrease of σ*_{d} with time can be justified by the dielectric behavior of cells at low frequencies (33). Upon increase of ρₒᵤₜ during evaporation, ions are squeezed into the cells, so that they become invisible to electric field, and therefore, the overall effect is as if the number of the existing ions for conduction has decreased.
(iii) With the increase of ρₜₒₜ, the external ionic concentration seen by each individual cell increases. Parallel to the discussion in part (ii), cells reduce their ion release due to effective increase of the osmotic pressure of their environment, Πₒᵤₜ.

To validate that activation of K⁺ osmoregulatory transporters is the main reason for uptake of ions as droplets evaporate, in one example, four different strains of S. typhimurium, WT, TrkA−, Kdp−, and the double mutant, TrkA−/Kdp− were studied. Cells with ρₜₒₜ˜107 cells per milliliter were resuspended in 1 μM KCl. The time-varying conductance results of cells are plotted in FIG. 6A. As shown, different strains show different responses to the continuous increase of K⁺ concentration because of droplet evaporation; more specifically, the TrkA− strains show the smallest rate of conductance increase. From these data, the ratio of the final per-cell conductivity to the initial value, σ_{f}*=σᵢ* was extracted, as plotted in FIG. 6B. The double-mutant samples lack both the primary K⁺ responders and hence, show the highest conductance (less K⁺ stealing from the external droplet). TrkA− mutant, which only has the Kdp transporter (the most selective K⁺ channel), steals the largest amount of K+ from the solution (i.e., has the smallest conductance). WT and Kdp− cells show almost similar responses, which is because both strains have the TrkA transporter, which is the first and main responder to the changes of K⁺ concentration (20). These results further confirm that evaporation-induced modulation of osmotic pressure because of increase of K⁺ concentration is the main mechanism underlying the time-dependent uptake of ions by cells.

There are several techniques for detection of bacterial viability, such as, colony counting, fluorescent staining, molecular-based methods (involving antibodies, DNA, or RNA), impedance microbiology, DEP-based differentiation (3, 18, 33), and light-addressable potentiometric sensors (LAPS). A comparison between the detection time versus cell concentration of the existing viability assays and the present work is illustrated in FIG. 7.

Although conventional microbiological methods, such as colony counting, are extremely sensitive, efficient, and inexpensive, their detection time not only increases exponentially as the cell concentration decreases, but also depends on bacteria type and how fast they multiply. Such methods, therefore, are not suitable for fast diagnosis in emergency cases. Among various automated, label-free viability platforms, impedance microbiology (IM) is promising because of simple device assembly/instrumentation and their integrability with the microelectronics technology. The IM technique involves monitoring the impedance changes of a pair of electrodes immersed in the growth medium. These changes are produced by release of ionic metabolites from live cells as they multiply. Similar to the colony counting method, the detection time of IM methods is quite long due to the lengthy cell incubation required for reaching a certain threshold signal. Therefore, as long as the sensing platform relies on cell growth, rapid viability detection is challenging, especially at low cell concentration. In this context, advantages of the presently disclosed incubation-free, osmoregulation-based approach can be substantial.

It shall be understood that while the above examples are related to viability of bacteria, differentiation of various bacteria types may also be evaluated using the disclosed process and apparatus. For example, a pre-growth step on a selective medium or an antibody-based filtering as a part of the detection protocol may be performed.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into the processor (and possibly also other processors), to cause functions, acts, or operational steps of various aspects herein to be performed by the processor. Computer program code for carrying out operations for various aspects described herein may be written in any combination of one or more programming language(s).

The invention is inclusive of combinations of the aspects described herein. References to “a particular aspect” or “embodiment” and the like refer to features that are present in at least one aspect of the invention. Separate references to “an aspect” (or “embodiment”) or “particular aspects” or the like do not necessarily refer to the same aspect or aspects; however, such aspects are not mutually exclusive, unless so indicated or as are readily apparent to one of skill in the art. The use of singular or plural in referring to “method” or “methods” and the like is not limiting. The word “or” is used in this disclosure in a non-exclusive sense, unless otherwise explicitly noted.

The invention has been described in detail with particular reference to certain preferred aspects thereof, but it will be understood that variations, combinations, and modifications can be effected by a person of ordinary skill in the art within the spirit and scope of the invention.

What is claimed is:

1. A method of determining a concentration of a biological material in a liquid sample, comprising:
electrically monitoring a liquid droplet whose conductivity is modulated over time by osmoregulation response of cells contained within the droplet to determine monitoring data, wherein said osmoregulation response is stimulated by evaporation of the droplet over time; and
comparing the monitoring data to calibration data in order to determine the concentration of the cells.

2. The method of claim 1, wherein the cells comprise bacteria.

3. The method of claim 1, wherein said concentration comprises concentration of live cells as compared to dead cells in the droplet.

4. The method of claim 3, wherein the cells comprise bacteria.

5. The method of claim 1, wherein said concentration comprises concentration of a first cell type as compared to a second cell type, wherein the first cell type and the second cell type have differing osmoregulation responses.

6. The method of claim 1, wherein said electrical monitoring comprises monitoring the impedance or conductance of the droplet using a first and second electrode, said first and second electrode in electrical contact with the droplet.

7. The device of claim 6, wherein said electrical monitoring comprises mapping the physical distribution of said cells within the droplet by monitoring the impedance or conductance of the droplet using multiple pairs of electrodes to map the physical distribution of said cells within the droplet.

8. A device for determining a concentration of a biological material in a liquid sample, comprising:
a first electrode;
a second electrode, the first and second electrode configured to pin a liquid droplet in a first contact area such that as the droplet evaporates, the contact area remains substantially constant; and
a monitoring unit operatively connected to the first and second electrodes, the monitoring unit configured to electrically monitor the droplet to determine monitoring data as conductivity of the droplet is modulated over time by osmoregulation response of cells contained within the droplet.

9. The device of claim 8, where said osmoregulation response is stimulated by evaporation of the droplet over time.

10. The device of claim 8, wherein the first electrode comprises an array of parallel elongated members mounted upon a substrate.

11. The device of claim 10, wherein the second electrode comprises an array of parallel elongated members mounted upon a substrate.

12. The device of claim 8, wherein the monitoring unit further compares the monitoring data to calibration data in order to determine the concentration of biological material in the droplet.

13. The device of claim 8, wherein the cells comprise bacteria.

14. The device of claim 8, wherein said concentration comprises concentration of live cells as compared to dead cells in the droplet.

15. The device of claim 8, wherein said concentration comprises concentration of a first cell type as compared to a second cell type, wherein the first cell type and the second cell type have differing osmoregulation responses.

16. The device of claim 8, wherein said electrical monitoring comprises monitoring the impedance or conductance of the droplet using the first and second electrode.

17. The device of claim 8, further comprising at least a third electrode electrically connected to the monitoring unit, wherein the monitoring unit is configured to electrically monitor the droplet using selected pairs of said electrodes to map the physical distribution of said cells within the droplet.
